(19) United States
(12) Patent Application Publication
(10) Pub. No.: US 2008/0306495 A1
(43) Pub. Date: Dec. 11, 2008

(54) Transilluminating laparoscopic ligating vascular clamp (LVC)

(76) Inventors: Ronald J. Thompson, Ft. Thomas, KY (US); Donald N. Halgren, Manchester, MA (US)

Correspondence Address: Donald N Halgren, 35 Central Street, Manchester, MA, 01944, US

(21) Appl. No.: 11/810,953

(22) Filed: Jun. 7, 2007

(51) Int. Cl. A61B 17/122

(52) U.S. Cl. 606/151

(57) ABSTRACT

A transilluminating laparoscopic ligating clamp (LVC) arrangement comprising an elongated generally “J” shaped dorsal clamp component and an elongated generally “J” shaped ventral clamp component, arranged to be receivably lockable with the dorsal clamp component about a living tissue element.

### FIELD OF THE INVENTION

The present invention relates to medical devices and more particularly to vascular clamps arranged to laparoscopically completely obstruct a large caliber blood vessel contained within a surgical pedicle of that blood vessel (artery) and its accompanying vein and supportative connective tissue.

### BACKGROUND OF THE INVENTION

Surgery, either approached via an open incision, such as a laparotomy or via the laparoscopic method, involves accomplishing a specific task without damaging surrounding vital structures. In cholecystectomy, gall bladder removal, the vital structure at most risk for injury is the common bile duct, the conduit for bile produced in the liver and channeled into the small intestines. In gynecologic procedures, the vital structure most commonly at risk for injury is the ureter, the muscular channel that drains urine from the kidney into the urinary bladder. Injuries to these vital structures can occur from a multitude of insults, including blunt trauma from dissection to identify both the vital structures themselves and the surrounding blood vessels, partial or complete obstruction of these conduit vital structures when physically occluding blood vessels, or thermal damage when using an electric current (RF) to occlude blood vessels.

Occluding blood vessels is the hallmark of excisional abdominal surgery, such as for cholecystectomy, hysterectomy or salpingo-oophorectomy. Blood vessels are not easily recognizable, even though they may be as large as one centimeter in diameter, the diameter of a large earthworm. The blood vessels are encased in adventitial consecutive tissue and peritoneum usually several millimeters or more thick to support the blood vessels. A thick walled artery, thin walled vein and accompanying nerves defines the neurovascular bundles, that is surrounded by adventitial connective tissue. Absolute dissection of the neurovascular bundle to gain perfect visualization of a skeletanized artery is likely to cause tearing and bleeding from the accompanying thin walled fragile view.

In cholecystectomy, careful partial dissection of the most superficial adventitial tissue to allow identification of the pulsing cystic artery is usually accomplished, to minimize vein damage. In gynecologic surgery, the infundibalopelvic ligament is not usually dissected to skeletonize the ovarian artery, but rather a pedicle is developed and ligated. (See definition.) Ovarian artery welding or sealing device manufacturers recommend skeletanization of the ovarian artery from the infundibalopelvic ligament to concentrate the RF energy directly on the ovarian artery to seal the artery.

This recommended ovarian artery skeletinization places the nearby ureter at risk of injury, but using RF energy without skeletanization of the ovarian artery places the ureter at risk of thermal injury. Ovarian artery skeltanization is recommended because the ovarian artery is at the uppermost limit of the size of arteries that can be safely or effectively sealed by RF energy.

Size matters. Small arteries can be effectively occluded by RF energy or externally compressing surgical clips. Moderately larger arteries (3 mm to 7 mm) can only be effectively occluded with suture ligation, RF sealing (welding) or by use of the stapler-cutter if preformed laparoscopically. Large vessels 7 mm to 12 mm can only be effectively occluded with suture ligation or use of the stapler-cutter; although the stapler-cutter is not specifically designed or FDA approved for arterial occlusion.

Major arteries greater than 12 mm are rarely occluded except in large segment bowel resections and limb amputations.

### DEFINITIONS PERTINENT TO THE PRESENT INVENTION

(1) Trans-illumination—using a focused beam of light arranged behind tissue to view the structures located within that tissue.
(2) Vessel Skeletanization—the surgical dissection of all of the adventitial tissue off of the blood vessels, to yield an addressable blood vessel for ligation or sealing.
(3) Vascular Pedicle—the result of a surgical procedure where the overlying adventitial tissue and the blood vessels contained within are clamped, cut downstream from the arterial blood flow, and suture ligated upstream of the clamp. The clamp is removed as the suture is tied tightly and the vascular pedicle is hemostatic (no bleeding).
(4) Surgical Clip (hemoclip)—an external occlusion of a skeletanized blood vessel by crimping a metal v-shaped clip onto itself, with the blood vessel contained within.
(5) Vessel Welding or Vessel Sealing—(thermal weld) a method to utilize an electric current to denature the proteins and dessicate the water in tissues, to seal the skeletanized blood vessels.
(6) Surgical Stapler—Cutter Device—a device intended for small bowel trans-sections that uses an anvil to deform titanium staples once passed through the two wall thickness of the small bowel. Six rows of staples are interspaced to prevent tissue necrosis, and the cutting element transsects the small bowel between the third and fourth rows of staples. This occlusion of the small bowel proximal to and distal to the trans-section prevents spillage of fecal material into the peritoneal cavity. The stapler-cutter has gained acceptance for the hemostatic ligation and transsection of the infundibulopelvic ligament in gynecologic surgery.
(7) Infundibulopelvic ligament—the most cephalad, lateral aspect of the broad ligament that contains the ovarian artery and ovaries vein.
(8) Salpingo-Oophorectomy—surgical excision of the ovary and fallopian tube.
(9) Risk Reducing (prophylactic) Salpingoophorectomy—surgical excision of both ovaries and both fallopian tubes in women who are at increased risk of developing ovaries or tubal cancer or who have occult cancer of the fallopian tube or ovary.

### PRIOR ART DISCUSSION

Prior Art for surgical clips (hemoclips) vessel welding and vessel sealing, and for surgical stapler-cutter devices are identified. Also referenced is prior art for the transillumination of vessels and other structures within tissues. U.S. Pat. No. 6,030,402 teaches of a transillumination device arranged within the peritoneal cavity and held adjacent to the most inferior aspect of the abdominal wall to transillumite the abdominal wall to allow insertion of a surgical trocar, to avoid injury to arteries within the abdominal wall. In this arrangement, the surgeon's vision is on the external surface of the abdominal skin.

### Ovarian Cancer

Fifty to seventy thousand women are newly diagnosed, in the United States, with ovarian cancer each year. The worldwide number of newly diagnosed women with ovarian cancer each year is unknown, but certainly a multiple of the US experience. Ovarian cancer is one hundred percent fatal. Ovarian cancer is an occult cancer. Webster defines occult in medical terms a “not readily detectable, especially at the place of origin.” Ovarian cancer is staged in four states, with the earliest states I and II, usually being found accidentally during an unrelated gynecologic surgery. Stage III and IV ovarian cancer have no pain, and patients usually present with several months of vague symptoms of abdominal fullness or abdominal distension, usually attributed by the patient to digestive difficulties.

Recently, ovarian cancer has been expanded to include distal fallopian tube cancer and peritoneal cancer. The reason that ovarian/tubal cancer is so difficult to diagnose and treat is that this cancer is considered a total peritoneal disease, especially at Stage III and Stage IV. The peritoneum is the lining of the abdominal cavity. State III and IV ovarian/tubal cancer is normally spread over the entire surface of peritoneal cavity, as the wind spreads the dandelion seed and over an entire yard. This seeding of the entire peritoneal surface is the etiology of the vague digestive type symptoms of Stage III and Stage IV ovarian/tubal cancer. This seeding of the entire peritoneal cavity is also the reason for the one hundred percent fatality rate of ovarian cancer.

Prevention of ovarian/tubal cancer is the only effective treatment for ovarian cancer. If every women had her ovaries and fallopian tubes removed surgically after her childbearing, the incidence of ovarian cancer would decrease dramatically, but this prevention is probably excessive. Identification of high risk women for ovarian/tubal cancer would allow the removal of her ovaries and fallopian tubes, a salpingo-oophorectomy, after childbearing. Currently, there are two identified groups at high risk for ovarian/tubal cancer, female relatives of women who have been diagnosed with ovarian/tubal cancer, and women with a BRCA1 or BRCA2 genetic mutation. These genetic mutations prevent normal cell repair of ovarian/tubal cell lines and are inherited. The BRCA represents Breast Cancer. Dr. Papic reports in 2006 “Funtional implications of BRCA1 for early detection, prevention and treatment of breast cancer,” Germline mutations in BRCA1 confer a 56-80% lifetime risk for breast cancer, and a 15-60% lifetime risk for ovarian cancer in women. Dr. Finch, et al. in a recent JAMA (Jul. 12, 2006) article “Salpingo-oophorectomy and the risk of ovarian, fallopian tube, and peritoneal cancers in women with BRCA1 or BRCA2 mutation reports, of 1828 women, “The overall (adjusted) reduction in cancer risk associated with bilateral oophorectomy is 80% (95% confidence interval, P=0.003).

More recently, in February 2007, article by Dr. CP Cran et al., “The distal fallopian tube: a new model for pelvic serous carcinogenesics” published in Current Opinions in Obstetrics and Gynecology concludes “The emerging data offer compelling evidence for a model of ‘Fimbrial-ovarian’ serous neoplasm, and call attention to the distal fallopian tube as an important source for this disease.” In 2006, Dr. Y Lee et al. also from Boston's Brigham and Women's Hospital (at Dr. Crum), “Prophylactic salpingo-oophorectomies from women with BRCA mutation have identified the tube as a prerequisite site of early pelvic serous carcinoma (tubal intraepithelial carcinoma [TIC]). This was reported in January 2006 journal Advances in Anatomical Pathology article entitled “Advances in the recognition of tubal intraepithelial carcinoma applications to cancer screening and the pathogensis of ovarian cancer.”

Therefore, to prevent ovarian/tubal cancer, it is important to identify the women at risk, and to reduce their risk of developing ovarian/tubal cancer with a prophylactic salpingo-oophorectomy surgical procedure. The problems with identifications of women at risk is that one or more first line female relatives must have suffered either breast cancer or ovarian cancer, and that the initial genetic identification for the BRCA1 and BRCA2 genetic mutations is an expensive diagnostic test, costing over three thousand dollars. If the locus of the genetic mutation of BRCA1 or BRCA2 is identified for one family member, all female relatives can be diagnosed with specific locus mutation for BRCA1 and BRCA2 for only two hundred dollars per individual. Numerous laboratories are attempting to develop not only more cost effective diagnostic tests for the BRCA1 and BRCA2 genetic mutation, but even cost effective screening tests for BRCA1 and BRCA2. In 1999, Dr. Van Orsoan et al. reported in the CAT.INST.PR article entitled “A highly accurate, low cost test for BRCA1 mutation that the cost of screening per sample was calculated to be approximately US $70 for the manual technique used in this study, and may be reduced to US $10 with the introduction of commercially available PCR robotics and fluorescent imaging. This wide spread availability of a cost-effective automated screening test for BRCA2 and BRCA2 genetic mutation is the first step to reduce the risk of ovarian/tubal cancers”. The second step to reduce the risk of ovarian/tubal cancers is to produce a safe, low-risk, cost-effective laparoscopic out-patient procedure for risk reducing prophylactic bilateral salpingo-oophorectomy.

### Laparoscopic Bilateral Salpingo-Oophrectomy

Each ovary and fallopian tube, referred by gynecologists as the adrexae, has a duel arterial blood supply, from the ovarian artery and from an ascending branch of the uterine artery. This ascending branch of the uterine artery is moderate in size, 3-4 mm, ascends for 4-6 cm in the broad ligament, just adjacent to the uterine boarder, and courses with a 70-900 turn at the junction of the uterine fundus and the fallopian tube. This artery then courses in the mesosalpinx just below the course of the fallopian tube and supplies the ovary. During a salpingo-oophorectomy procedure, the fallopian tube is obstructed and ligated at its junction with the uterus, and the ascending branch of the uterine artery is also obstructed and ligated. Because the fallopian tube in this proximal isthmic region is quite muscular, and 1 to 1.5 cm thick, a hemoclip or surgical clip is not appropriate for surgical obstruction of the muscular fallopian tube and the artery that lies just ventral to the fallopian tube. Either electrocautery or a surgical stapler-cutter is currently employed to obstruct and transsect both the fallopian tube, at its junction with the uterine fundas, and the ascending branch of the uterine artery, in the ovarian ligament.

The ovarian artery is the major blood supply to the ovary and the distal fallopian tube. The ovarian artery arises from the anterior surface of the aorta just below the level of the renal arteries. The ovarian artery is large, 6 to 10 mm or more in diameter, and has a strong pulse pressure because it is a direct branch of the aorta. This is the largest concern of gynecologists when performing a salpingo-oophrectomy. In fact, gynecologic operative textbooks describe double ligating the ovarian artery with two separate sutures during a hysterectomy or salpingo-oophrectomy to reduce the risk of post-operative bleeding from the ovarian artery pedicle. This is the greatest risk of any salpingo-oophorectomy procedure, post-operative ovarian pedicle bleeding. Exsanquination can occur in fifteen to thirty minutes. Currently, the surgical stapler-cutter is utilized to obstruct and transect the infundibulopelvic ligament that contains the ovarian artery, during laparoscopic salpingo-oophorectomy. Surgeons will laparoscopically double tie the infundibulopelvic ligament if a suturing technique is used. Few, if any, surgeons will use the RF electrocautery welding or sealing of the skeletanized ovarian artery or the infundibulopelvic ligament, because of the risk of postoperative bleeding.

### Concerns of Current Technology for Outpatient Risk Reducing Laparoscopic Salpingo-Oophorectomy.

LigaSure™ vessel sealing system is currently marketed by Valleylab, a division of Tyco Healthcare. In the LigaSure™ marketing brochures, the LigaSure™ is recommended for sealing arteries “up to and including 7 mm in diameter.” The ovarian artery can be 8 or 9 mm in diameter, and therefore, not included in the upper limits of efficiency for the LigaSure™ vessel sealing system. In addition, since the ovarian artery has a high pulse pressure during systole, because of its direct branching of the aorta, a “blow-out” of the sealed ovarian artery is of great concern. These “blow-outs” would not be expected intraoperatively or in the two hours of the immediate post-operative recovery period, but after six to twelve hours post vessel sealing, when the patient is at home and not medically observed. Therefore, not only is the LigaSure™ vessel sealing system not appropriate for ligation of the ovarian artery, it is not appropriate in an outpatient setting. Once again, exsanquination can occur in fifteen to thirty minutes after failure of any ligation method for the ovarian artery.

Another reason not to utilize the vessel sealing during a risk reducing prophylactic salpingo-oophorectomy is that the electrical energy used for the vessel sealing might cause thermal damage to the specimen to be histologically examined for signs of occult cancer.

In “Clinical outcome of prophylactic oophorectomy in BRCA1/BRCA2 mutation carriers and events during follow-up” published by Dr. RI Olivier et al. in the British Journal & Cancer, 2004 April 19:90(8), Dr. Olivier reports “at the time of salpingo-oophorectomy, five of 58 BRCA1 carriers (8.6%) were diagnosed with occult carcinoma: two fallopian tube carcinomas, two ovarian carcinomas and one case was defined as a fallopian tube/ovarian carcinoma.” Thermal damage to the specimen would prevent the post-operative diagnosis of an occult fallopian tube/ovarian carcinoma and therefore prevent a patient from the appropriate post-operative chemotherapy that would extend her life for years.

### Ovarian Artery Stapling-Cutting

Although currently utilized to staple and transsect the ovarian artery within the infandibulopelvic ligament during laparoscopic salpingo-oophorectomy, the linear staler-cutter has many shortcomings. The linear stapler-cutter device is very expensive, adding from $1,000 to $1,500 per case depending upon the number of cartridges used. The linear cutter-stapler is a very complex device and the FDA med-watch device-malfunction reporting service lists over one thousand documented instances of device malfunction. Reports include:
(1) Device was applied during a oophorectomy procedure. The instrument misfired.
(2) During a laparoscopically assited vaginal hysterectomy procedure, the staples did not form properly.
(3) Device was applied during an endoscopic procedure. The instrument would not open after firing.

This last report is the most concerning relative to the complexity of the stapler-cutter, for to remove the instrument from the infundibulopelvic ligament would require the conversion to a laparotomy and a three-day hospital stay and at least a four week post-operative recovery for what was intended as an out-patient surgery.

In addition, the use of the stapler-cutter instrument places three lines of titanium staples in the specimen to be removed. The presence of the titanium staples not only complicates the removal of the excised fallopian tube and ovary from the pneumoperitoneum through a 10 mm or 12 mm cannula, they also interfere with the post-operative tissue preparation for histologic examination. This interference with the histological examination could contribute to the missed diagnosis of an occult fallopian tube or ovarian cancer in these women at high risk for cancer, as reported by Dr. Olivier in the article previously cited. Therefore, although correctly used for ligation of the ovarian artery in laparoscopically accomplished salpingo-oophorectomy procedures, the linear stapler-cutter device has many shortcomings, including expense, device malfunction and the presence of titanium staples in the excised operative specimen.

The invention thus comprises a transilluminating laparoscopic ligating clamp (LVC) arrangement comprising: an elongated generally “J” shaped dorsal clamp component; an elongated generally “J” shaped ventral clamp component, arranged to be receivably lockable with the dorsal clamp component about a living tissue element. The dorsal clamp component and the ventral clamp component are in longitudinal alignment with one another. The dorsal clamp component has a ventral clamp component locking means arranged both proximally and distally thereon. The ventral clamp component has a dorsal clamp component locking means arranged both proximally and distally thereon. The dorsal clamp component has a sharp tissue piercing distalmost tip end. The ventral clamp component has a tissue piercing tip receiving channel therein. The dorsal clamp component and the ventral clamp component each have a mateable alignment of alignment members thereon to insure tight mating of said components during piercing of tissue therebetween. The dorsal clamp component and the ventral clamp component are light transmissive to permit visual examination of tissue pierced therebetween. A light emitter is preferably arranged adjacent the clamp components.

The invention also includes a method of ligating a vessel comprising one or more of the following steps of: introducing an elongated ventral clamp component in to a tissue supportive orientation; introducing an elongated dorsal clamp component into a parallel, tissue ligating relationship with the ventral clamp component; and illuminating the tissue ligating relationship with an articulable light source adjacent one of the clamp components; locking the dorsal clamp component into the ventral clamp component to effect ligating of the tissue. The dorsal clamp component and the ventral clamp component may each have a pair of mateable interlocking components therewith. The method may include guiding the dorsal clamp component into the ventral clamp component by an alignment guide arranged on each of the clamp components; introducing the ventral clamp component into a supportive tissue engaging orientation, longitudinally followed by introduction of the dorsal clamp component nestingly thereagainst, in a tissue-piercing, interlocked relationship; visualizing the illuminated tissue ligating relationship of the clamp components by a visualizing scope.

### DESCRIPTION OF THE DRAWINGS

The objects and advantages of the present invention will become more apparent when viewed in conjunction with the following drawings in which:

FIG. 1 represents a side elevational view of a two piece ligating vascular clamp in a spaced-apart, vessel pre-clamping configuration;

FIG. 2 is a view showing the clamp depicted in FIG. 1 in a vessel contacting configuration;

FIG. 3 represents the clamp shown in FIG. 2 as the clamp pierces the ligament;

FIG. 4 represents the clamp shown in FIG. 3, with the clamp fully closed and locked upon the pierces ligament; and

FIG. 5 is a view taken along the lines 5-5 of FIG. 1.

### DESCRIPTION OF THE PRESENT INVENTION

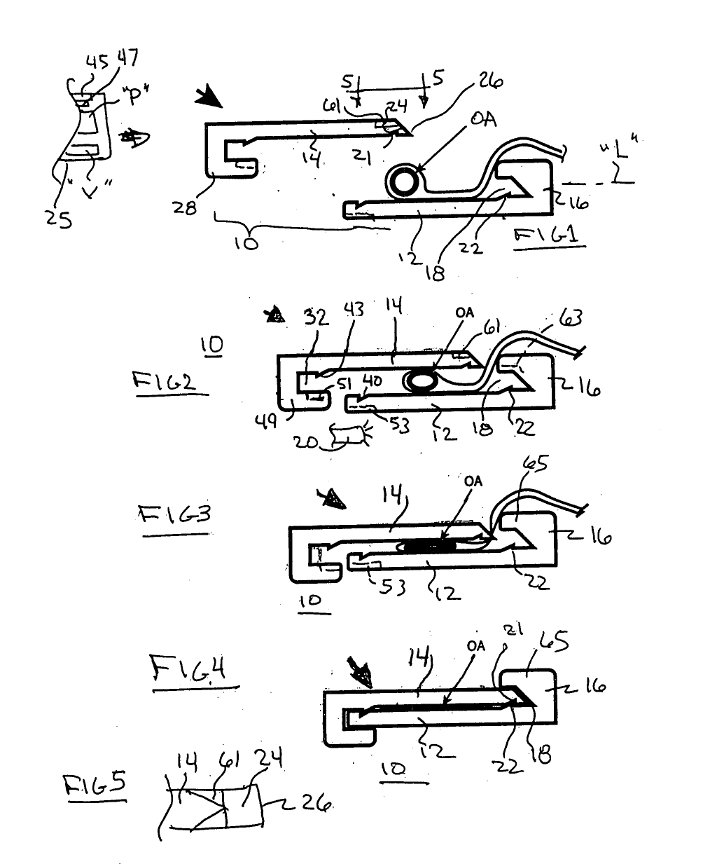

Referring now to the drawings in detail, and particularly to FIG. 1, there is shown the Transilluminating Laparoscopic Ligating Vascular Clamp (LVC) 10. The clamp 10 is a two-piece clamp to laparoscopically completely obstruct a large caliber blood vessel, (such as for example, an ovarian artery OA) contained within a surgical pedicle of that blood vessel (artery) and its accompanying vein and supportative connective tissue, as though the surgical pedicle was ligated with a hand tied suture, or two sutures. The Ligating Vascular Clamp 10 is composed of a ventral clamp component 12 and a dorsal clamp component 14, as shown in a pre-clamping, spaced-apart configuration in FIG. 1.

Both clamp components 12 and 14 are capable of transmitting light because of their composition, such as clear LEXAN. The two clamp components are not attached to one another, but are introduced serially, through the scope 25, as represented in FIG. 1, until their final aligned, locked mating of the dorsal clamp component 14 to the ventral clamp component 12, both distally and proximally, as represented in FIG. 4.

Ventral Clamp Component

The ventral clamp component 12 of the Transilluminating Laparoscopic Ligating Vascular Clamp 10 is for example, preferably between about 20 and 40 mm long, about 8 to 12 mm wide, about 2 to 4 mm thick and at the distal and has an elevated 90 degree upward, generally “J” shaped projection 16, at about 8 to 12 mm high and the relative width of the entire ventral clamp component 12. The distal elevated projection 16 contains a receiving channel 18 to alignably and lockingly receive the distal dorsal clamp component 14 with a one way locking mechanism 22 to distally note the dorsal clamp component 14 to the ventral clamp component 12. The elevated projection 16 of the ventral clamp component 12 acts like a tissue retractor to elevate the broad ligament beyond the infundibulopelvic ligament when positioned beneath the infundibulopelvic ligament. During this intended laparoscopic maneuver, the ventral clamp component 12 of the clamp device 10 is firmly seated and attached to the distal end of a 30-35 cm laparoscopic application device 25. The dorsal clamp component 14 of the Transilluminating Laparoscopic Ligating Vascular Clamp 10 is firmly sequestered within the distal aspect of the laparoscopic application device 25 just proximal to the ventral clamp component 12. With the dorsal clamp component 14 sequestered and hidden, there is complete unobstructed laparoscopic vision of the dorsal aspect of the infundibulopelvic ligament and the broad ligament.

Transillumination

With unobstructed dorsal vision of the infundibulopelvic ligament and the broad ligament, the ventral clamp component 12, when positioned beneath the broad ligament beyond the infundibulopelvic ligament, will transilluminate all of the structures within the infundibulopelvic ligament when a light source 20, represented in FIG. 2 abutts the proximal ventral clamp component 14.

The transilluminating light source 20 will originate from a standard source, be fiber-optically transmitted to the proximal aspect of the laparoscopic application device 25, and along fiberoptic fibers contained within the shaft of the laparoscopic application device 25 to terminate abutting the proximal aspect of the ventral clamp component 12. This transilluminating light of the ventral clamp component 12 will allow visual identification by a visualization device “V” of the ovarian artery OA contained within the tissues of the infundibulopelvic ligament IL, and more importantly to visually insure the absence of the ureter, where the clamp 10 is positioned, before the dorsal clamp component 14 is advanced and the infundibulopelvic ligament ligated. The ovarian artery and the ureter are both contained within the broad ligament, but the ureter is usually much more laterally placed, retroperitoneally.

Dorsal Clamp Component

The dorsal clamp component 14 of the Transilluminating Laparoscopic Ligating Vascular Clamp 10 is composed of the same material as the ventral clamp component 12 and is designed to be almost an inverted mirror image of the ventral clamp component 12, to allow distal and proximal mating and locking of the two clamp components 12 and 14, as represented in FIGS. 2-4. The dorsal clamp component 12 is the same length, width, and thickness as the ventral clamp component 14. The distal end 24 of the dorsal clamp component 14 has a sharpened tip 26 to puncture the anterior and posterior leaves of the broad ligament beyond the infundibulopelvic ligament, as represented in FIG. 3. This puncture of the broad ligament is facilitated by the counter-traction of the broad ligament by the distal upward 90 degree generally “J” shaped projection 16 on the distal ventral clamp component 12, and the channel 18 defined by that upward projection 16. With the broad ligament stretched across the receiving channel 18, as represented in FIG. 2, the distal end 24 of the dorsal clamp component 14 can easily penetrate the broad ligament and lock distally to the ventral clamp component 12. Simultaneous to the distal ratcheting locking of the dorsal clamp component 14 to the ventral clamp component 12, the generally “J” shaped proximal portion 28 of the dorsal clamp component 14 mates with the proximal aspect 30 of the ventral clamp component 12. The proximal portion 28 dorsal clamp component 14 has its generally “J” shaped 90 degree downward projection with a female receptacle or receiving channel 32 to accept the male end 30 of the proximal ventral clamp component 12 upon mating of the dorsal clamp component 14 to the ventral clamp component 12, as represented in FIG. 4. This mating at the proximal end of each of the clamp components 12 and 14 is lockably finalized by a ratcheting locking mechanism 40 and 43, and 21 and 22.

### Parallel Mating of the Dorsal

Clamp Component to the Ventral Clamp Component

The dorsal clamp component 14, initially sequestered within the distal thread of the laparoscopic application device 25, is advanced forward with the forward movement of a plunger that extends the entire length of the application device 25. The internal aspect of the distal application device or scope 25 has two grooves 45 and 47, that house multiple lateral projections on either side of the dorsal clamp component 14. These internal surface grooves slope at about 30 degrees to the central longitudinal axis “L”, so that a forward plunger motion causes the dorsal clamp component to close parallel to the ventral clamp component 12 as an airplane closes to the ground upon landing. The continued forward plunger motion, once the distal and proximal locking mechanisms 21/22 and 40/43 mate, causes a tightening and obstruction of the tissues positioned between the ventral and dorsal clamp components 12 and 14. The proximal end 49 of the dorsal clamp 14 has a tapered alignment flange 61 which closingly and alignably mates with a correspondingly shaped alignment notch 63, represented in FIG. 5 and FIG. 3. The distal end of the dorsal component 14 has a tapered flange 61 which closingly and alignably mates with a correspondingly shaped receiving notch 63 in the tip 65 of the receiving channel 18 to provide longitudinal alignment of the components 12 and 14 as they close-in upon one another preceding their being locked together. The ratcheting of the distal and proximal locking mechanisms 22, and 40 and 42 may allow tactile and/or audible feedback to the surgeon while advancing the plunger “P” within the application device 25. This forward motion will range 30 to 40 mm and the complete ligating vascular clamp 10 closure will be accomplished in seconds.

1. A transilluminating laparoscopic ligating clamp (LVC) arrangement comprising:
an elongated generally “J” shaped dorsal clamp component;
an elongated generally “J” shaped ventral clamp component, arranged to be receivably lockable with said dorsal clamp component about a living tissue element.

2. The LVC as recited in claim 1, wherein said dorsal clamp component and said ventral clamp component are in longitudinal alignment with one another.

3. The LVC as recited in claim 1, wherein said dorsal clamp component has a ventral clamp component locking means arranged both proximally and distally thereon.

4. The LVC as recited in claim 1, wherein said ventral clamp component has a dorsal clamp component locking means arranged both proximally and distally thereon.

5. The LVC as recited in claim 1, wherein said dorsal clamp component has a sharp tissue piercing distalmost tip end.

6. The LVC as recited in claim 5, wherein said ventral clamp component has a tissue piercing tip receiving channel therein.

7. The LVC as recited in claim 1, wherein said dorsal clamp component and said ventral clamp component each have a mateable alignment of alignment members thereon to insure tight mating of said components during piercing of tissue therebetween.

8. The LVC as recited in claim 1, wherein said dorsal clamp component and said ventral clamp component are light transmissive to permit visual examination of tissue pierced therebetween.

9. The LVC arrangement as recited in claim 1, including a light emitter arranged adjacent said clamp components.

10. A method of ligating a vessel comprising:
introducing an elongated ventral clamp component in to a tissue supportive orientation;
introducing an elongated dorsal clamp component into a parallel, tissue ligating relationship with said ventral clamp component; and
illuminating said tissue ligating relationship with an articulable light source adjacent one of said clamp components.

11. The method as recited in claim 10, including:
locking said dorsal clamp component into said ventral clamp component to effect ligating of said tissue.

12. The method as recited in claim 11, wherein said dorsal clamp component and said ventral clamp component each have a pair of mateable interlocking components therewith.

13. The method as recited in claim 10, including:
guiding said dorsal clamp component into said ventral clamp component by an alignment guide arranged on each of said clamp components.

14. The method as recited in claim 10, including:
introducing said ventral clamp component into a supportive tissue engaging orientation, longitudinally followed by introduction of said dorsal clamp component nestingly thereagainst, in a tissue-piercing, interlocked relationship.

15. The method as recited in claim 19, including:
visualizing said illuminated tissue ligating relationship of said clamp components by a visualizing scope.
